(19) United States
(12) Patent Application Publication
(10) Pub. No.: US 2022/0117511 A1
(43) Pub. Date: Apr. 21, 2022

(54) DEVICE, SYSTEM AND METHOD FOR MONITORING A SITE OF INTEREST INTERNAL TO A PATIENT BODY

(71) Applicant: EXERO MEDICAL LTD, Or Yehuda (IL)

(72) Inventors: Erez SHOR, Kiryat Ono (IL); Shai POLICKER, Tenafly, NJ (US); Matan BEN DAVID, Tel Aviv (IL); Amir KRAITZER, Herzeliya (IL); Tal YERED, Pardes Hana Karkur (IL)

(21) Appl. No.: 17/261,971

(22) PCT Filed: Jul. 22, 2019
(86) PCT No.: PCT/IB2019/056247

§ 371 Date: Jan. 21, 2021

Related U.S. Application Data
(62) Division of application No. PCT/IB2018/058214, filed on Oct. 22, 2018.
(60) Provisional application No. 62/702,007, filed on Jul. 23, 2018.

(51) Int. Cl. A61B 5/0538; A61B 5/00

(52) U.S. Cl. CPC A61B 5/0538; A61B 5/0031; A61B 5/6852; A61B 5/7275

(57) ABSTRACT

A system and method for early detection of adverse physiological phenomenon such as anastomotic leakage through a tissue-of-interest monitoring electrode implanted in proximity to a site of interest internal to the body. The tissue-of-interest monitoring electrode is operative provide an electrical signal responsive to changes in physiological conditions at the site of interest. The electrical signals may be provided to by a communication device to a monitor which comprises circuitry that is operative to process the received signals for determining, for example, if GI leakage occurs or not. If leakage is detected, the monitor issues a warning indicating that leakage occurs.

### BACKGROUND

Monitoring physiological processes following surgery is critical to ensure complications are addressed as soon as possible for proper recovery, healing and/or to provide guidance for personalized treatment. Anastomotic leak, for example, is a common, life threatening complication in gastrointestinal (GI) surgeries. Early detection is essential for proper treatment and complete recovery. Current methods of detecting anastomotic leak are either delayed, inaccurate or invasive and may compound patient trauma. Therefore, there is a need for providing efficient alternative systems and methods providing early anastomotic leak detection.

### BRIEF DESCRIPTION OF THE DRAWINGS

The figures illustrate generally, by way of example, but not by way of limitation, various embodiments discussed in the present document.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. References to previously presented elements are implied without necessarily further citing the drawing or description in which they appear. The number of elements shown in the Figures should by no means be construed as limiting and is for illustrative purposes only. The figures are listed below.

FIG. 1 is a schematic block-diagram depiction of a physiological phenomenon detection sequence employable by the monitoring system, according to an embodiment;

FIGS. 2A and 2B depict a monitoring system coupled to a patient, according to various embodiments;

FIG. 3 is a schematic block-diagram depiction of a physiological phenomenon detection sequence employable by the monitoring system, according to an alternative embodiment;

FIGS. 4A to 4C are schematic depictions of various deployment options for deploying a sensing (also: monitoring) electrode into a patient body, according to embodiments;

FIGS. 5A-B and 5C-D are schematic depictions of different stages for operably deploying and fastening a monitoring electrode of the monitoring system onto patient tissue, according to an embodiment;

FIG. 6 is a flowchart of a method for monitoring a patient site of interest, according to some embodiments;

FIG. 7 shows an impedance plot of a threshold-based leak detection method;

FIG. 8 shows a plot of impedance measurement in induced leak;

FIG. 9 shows a plot of impedance measurement in sutured leak;

FIG. 10 shows representative electrophysiological signals measured by implanted electrodes; and

FIG. 11 shows cross correlation value (correlation coeff) of leak animal model vs. control animal model over time.

### DETAILED DESCRIPTION

Aspects of the present invention pertain to monitoring system employing an electronic sensor comprising one or more monitoring and, optionally, reference electrodes.

The leakage detection system may further comprise one or more communication devices comprising a transmitter, a receiver, and/or a transceiver (for implementing wired and/or wireless communication).

The communication device can be in communication with an external monitor and the electronic sensor. Optionally, a communication device may be external to the patient body for receiving and/or transmitting signals generated within the patient body and/or for receiving and/or transmitting signals generated outside the patient body. Optionally, a communication device may be an implantable communication device for receiving from and/or transmitting signals outside the patient body while implanted in the patient body via wired and/or wireless communication links. Optionally, a plurality of implantable communication devices implanted within the patient body may communicate with each other via wired and/or wireless communications.

Wired connections may be removable from a mammalian body (also: patient body) through a port and, as such, may be made or include nonbiodegradable, partially or fully biodegradable conductive material for the implementation of wired connections.

Optionally, a part of the electronic sensor may be biodegradable and/or biocompatible, and another part of the electronic sensor may be non-biodegradable and/or non-biocompatible.

Optionally the electronic sensor and/or the communication device components which are in contact with tissue of a patient site of interest (e.g., at a monitoring and/or reference location site) may be constructed, fully or partially, of biocompatible material and may, optionally, be partially or fully biodegradable. Optionally, non-biocompatible components may be housed within a sealed casing. Optionally, the term biodegradable may encompass the meaning of the term “bioresorbable” or “biodegradable yet non-bioresorbable”.

A reference electrode may be operably engagable with a site of interest (SOI) (e.g., internal organ tissue) of the patient at a location which is different from the location of the site of interest to which the tissue-of-interest monitoring electrode is operably deployed, to provide an output that can be used as reference to the output(s) provided by the tissue-of-interest monitoring electrode. For example, the sensing or monitoring electrode may operably engage a surgical site (e.g., a tissue connection site such as, for example, an anastomosis site), and the reference electrode may operably engage a patient site of interest at a location which is remote from the surgical site. For example, the reference electrode may operable engage the patient site of interest at a location at which no anastomotic leak is expected to occur. The two different patient site of interest locations may herein be referred to as “monitoring location site”, (or simply: “monitoring site”), to designate, for example, a surgical location site” (or simply: “surgical site”) and “reference location site” (or simply: “reference site”). The monitoring and/or reference location sites may be internal and/or external to the patient body. The monitoring and/or reference electrodes may be implantable or non-implantable monitoring and/or reference electrodes, respectively. Optionally, the reference electrode may be located external to the patient body.

The monitoring electrode may be implantable, biocompatible and, optionally, biodegradable in full or in part. In some embodiments, only the tissue-of-interest monitoring electrode is biodegradable in full or in part whereas the reference electrode may be non-biodegradable yet biocompatible. Optionally, a monitoring electrode referred to herein can be a multi-electrode or comprise an arrangement of multiple electrodes. Reading multiple signals from a plurality of electrodes enables better localization of the physiological phenomena and thus allow spatial or temporal-spatial monitoring of the signals, providing for example an indication indicates propagation velocity if the condition further develops. In some embodiments, a monitoring electrode can be referred to as an electrochemically responsive sensing or monitoring electrode.

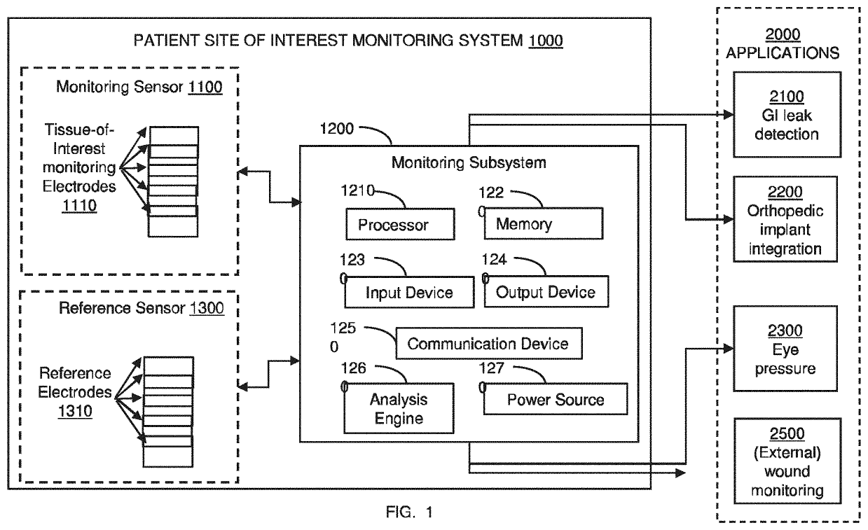

Referring now to FIG. 1, a patient site of interest monitoring system 1000 comprises a monitoring sensor 1100 comprising at least one monitoring electrode that is communicably coupled with a monitoring subsystem 1200. The patient site of interest monitoring system 1000 may in some embodiments also include a reference sensor 1300 comprising at least one reference electrode 1310 that is communicably coupled with monitoring subsystem 1200.

According to some embodiments, monitoring subsystem 1200 may include a processor 1210, a memory 1220, an input device 1230, an output device 1240, a communication device 1250, an analysis engine 1260, and a power module 1270 for powering the various components of patient site of interest monitoring system 1000 for the implementation of various applications 2000.

The various components of patient site of interest monitoring system 1000 may communicate with each other over one or more communication buses (not shown) and/or wired and/or wireless communication links.

Monitoring subsystem 1200 may be operatively coupled with monitoring sensor 1100 so that changes of electrical properties of monitoring electrodes 1110 are measurable by monitoring subsystem 1200, as outlined herein below in greater detail.

Monitoring subsystem 1200 may be operable to enable the implementation of a method, process and/or operation for allowing, for example, the detection of leakage from the lumen of an organ through an organ wall. Such method, process and/or operation may herein be implemented by an “analysis engine” of monitoring subsystem 1200, referenced by alphanumeric label “1260”. Analysis engine 1260 may be realized by one or more hardware, software and/or hybrid hardware/software modules, e.g., as outlined herein. A module may be a self-contained hardware and/or software component that interfaces with a larger system and may comprise a machine or machines executable instructions. For example, a module may be implemented as a controller programmed to, or a hardware circuit comprising, e.g., custom VLSI circuits or gate arrays, off-the-shelf semiconductors such as logic chips, transistors, or other discrete components, configured to cause patient site of interest monitoring system 1000 to implement the method, process and/or operation as disclosed herein. A module may also be implemented in programmable hardware devices such as field programmable gate arrays, programmable array logic, programmable logic devices or the like. For example, memory 1220, may include instruction which, when executed e.g. by processor 1210, may cause the execution of the method, process and/or operation for enabling, for example, the detection of leakage from a GI tract of the patient.

In some examples, analysis engine 1260 may be operable to supply artificial intelligence (e.g., machine learning functionalities) for determining if leakage occurs or not. Artificial intelligence and machine learning functionalities may be implemented, for example, by employing supervised and/or unsupervised machine learning using Support Vector Machine and/or an Artificial Neural Network. For example, a Support Vector Machine may be trained with a vector representing, for instance, 2-15 min of impedance measurement data and, optionally, with dimensionality reduced additional electrophysiological signals. For instance, the Support Vector Machine may be trained with electrophysiological signals (e.g., electrogastrography signals and/or other electro physiological signals recorded from the site of interest), for example, in the frequency domain and from which phase shifts are removed and/or which are otherwise processed to reduce dimensionality. In some embodiments, electro-physiological data representing phase, and/or phase shifts over time and/or phase differences between the signals recorded from the reference site and the signals recorded from the monitoring site may also be used as an input to analysis engine 1260.

Power module 1270 may comprise an internal power supply (e.g., a rechargeable battery) and/or an interface for allowing connection to an external power supply.

Input device 1230 of monitoring subsystem 1200 may be communicably coupled with monitoring electrodes 1110, e.g., in a wired or wireless manner to allow subjecting the patient site of interest, via one or more electrode of monitoring sensor 1100, with an input signal for stimulating the patient site of interest and, concurrently, measuring the voltage drop between at least two different locations of the same electrode(s). Monitoring subsystem 1200 may in some embodiments also be operable to sense an electrophysiological signal (e.g., electrogastrography and/or other electrophysiological signals) recorded by operably engaging electrodes with tissue of a patient site of interest. For example, specific variance characteristics (e.g., patterns) of EGG and/or other electrophysiological signals may be indicative of increased likelihood of onset of anastomotic leak and/or provide indication of an anastomotic leak occurring.

Input signals provided by input device 1230 may be direct current (DC) or alternating current (AC) signals. AC input signals may be provided at an input signal frequency ranging, for example, from 1 to 120 Hz. In some embodiments, frequency sweeping may be employed, and the output may be based on analysis of the response signal obtained over the swept input signal frequency range. Alternatively, step function input signals may also be used to obtain impedance measurement.

In some embodiments, signal processing may be employed for sensing or measuring impedance as well as, for example, for sensing a signal descriptive of electrophysiological activity of the tissue with which the electrode is operable engaged. An impedance signal may be received or sensed responsive to subjecting the electrode to an input (also: stimulation) signal. The frequency of the impedance signal corresponds with the frequency of the stimulation signal. The frequency of the stimulation signal may be selected such that the frequency of the obtained response signal (also: impedance signal) is outside the range of the frequency of the electrophysiological signal of interest. This way, the response signal and the electrophysiological signal can be separated from one another using signal filtering to allow concurrent sensing or measurement of the response signal and the electrophysiological signal using the same tissue-engaging electrode. For example, the frequency of the stimulation signal may be selected to range from 20 to 40 Hz and include, for example, 31 Hz resulting in response signals having frequencies in the range of 20 to 40 Hz, while the frequency of the electrophysiological signal may range from 0 to 12 Hz. Analysis engine 1260 may separate the signal components using signal filtering to allow for separate interpretation of the superpositioned response and electrophysiological signal components. For example, a notch filter may filter out the frequency of 31 Hz of impedance signals for allowing interpretation thereof, and a low-pass filter may be employed for filtering out a frequency range of 0-12 Hz for allowing interpretation of electrophysiological signals descriptive of, for example, GI activity.

In some embodiments, separate electrodes may be employed for measuring different types of signals such as impedance signals and electrophysiological signals

Electrical characteristics of input signals are controlled by analysis engine 1260 so that the magnitudes of electrical energy in the mammalian body are within physiologically tolerable values. A physiologically tolerable value may be, for example, an alternating current of 5 nA to 800 μA at a frequency range of 5-120 Hz.

Generally, impedance measurements may be sufficient to reliably estimate tissue condition and status. Electro-physiological signals may also be sufficient to monitor certain physiological conditions. The system may record impedance and electrophysiological information simultaneously, allowing flexibility for analysis engine 1260 which can be implemented using only a sub set of the signals (e.g., to allow comparatively lower computational complexity and smaller training datasets), or using all recorded data for enabling, for example, better sensitivity and faster detection.

Monitoring sensor 1100 may be operably coupled with the patient site of interest so that a sufficiently significant change in the material properties of monitoring electrodes 1110 causes a change in an electrical property of the monitoring electrode which is measurable and analyzable by analysis engine 1260. Information indicative of a detection in a change in the electrical property of monitoring electrodes 1110 may be conveyed to a user (not shown) via output device 1240. In some embodiments, analysis engine 1260 may be configured to cause output device 1240 to display values (e.g., auditory and/or visually) of the electrical properties as a function of time, e.g., within a calibrated scale, and/or provide an output of the analysis performed by analysis engine 1260 (e.g., provide an audible and/or visual alert).

In some embodiments, communication device 1250 may be equipped with a transmitter (not shown) and, optionally, with a receiver and/or a transceiver, e.g., for allowing the transmission of inputs or stimulation signals to monitoring electrodes 1110 and, optionally, to reference electrodes 1310. Analysis engine 1260 may control the generation of the input signals.

In some embodiments, communication device 1250 enables the transmission of response signals carrying data (“electric-property-data”) that is descriptive of a change of the electrical properties of monitoring sensor 1100 from monitoring electrodes 1110 to analysis engine 1260.

In some embodiments, electrophysiological (e.g., electrogastric) signals may be transmitted via communication device 1250 to analysis engine 1260 for the analysis thereby.

It is noted that although components of patient site of interest monitoring system may be illustrated as being implemented by a single component, this should by no means be construed in a limiting manner. For example, components of patient of site of interest monitoring system can be deployed to be executed on one site or distributed across multiple sites and operably interconnected. For instance, separate processors and memories may be allocated to analysis engine 1260, and separate communication devices may be implemented for implementing communication device 1250.

It is noted that in some embodiments, one or more components of monitoring subsystem 1200 may be internal and one or more components may be external to the mammalian body. For example, certain processors, memories, input devices, communication devices and/or our power sources may be internal to the mammalian body and certain processors, memories, input devices, communication devices and/or our power sources may be outside the mammalian body.

For example, communication device 1250 may be coupled with or include a transmitter (not shown) that may be operably positionable within mammalian body. Optionally, electric-property-data may be transmitted from within the mammalian body to the outside of mammalian body wired and/or wirelessly over communication link (not shown) for further analysis by analysis engine 1260.

In embodiments, some of monitoring subsystem 1200 functionalities may be implemented by a multifunction mobile communication device also known as “smartphone”, a personal computer, a laptop computer, a tablet computer, a server (which may relate to one or more servers or storage systems and/or services associated with a business or corporate entity, including for example, a file hosting service, cloud storage service, online file storage provider, peer-to-peer file storage or hosting service and/or a cyberlocker), personal digital assistant, a workstation, a wearable device, a handheld computer, a notebook computer, a vehicular device, a stationary device and/or a home appliances control system.

The term “processor” as used herein may additionally or alternatively refer to a controller. Such processor may relate to various types of processors and/or processor architectures including, for example, embedded processors, communication processors, graphics processing unit (GPU)-accelerated computing, soft-core processors and/or embedded processors.

According to some embodiments, memory 1220 may include one or more types of computer-readable storage media. Memory 1220 may include transactional memory and/or long-term storage memory facilities and may function as file storage, document storage, program storage, or as a working memory. The latter may for example be in the form of a static random access memory (SRAM), dynamic random access memory (DRAM), read-only memory (ROM), cache or flash memory. As working memory, memory 1220 may, for example, process temporally -based instructions. As long-term memory, memory 1220 may for example include a volatile or non-volatile computer storage medium, a hard disk drive, a solid state drive, a magnetic storage medium, a flash memory and/or other storage facility. A hardware memory facility may for example store a fixed information set (e.g., software code) including, but not limited to, a file, program, application, source code, object code, and the like.

Communication device 1250 may for example include I/O device drivers (not shown) and network interface drivers (not shown). A device driver may for example, interface with a keypad or to a USB port. A network interface driver may for example execute protocols for the Internet, or an Intranet, Wide Area Network (WAN), Local Area Network (LAN) employing, e.g., Wireless Local Area Network (WLAN)), Metropolitan Area Network (MAN), Personal Area Network (PAN), extra net, 2G, 3G, 3.5G, 4G including for example Mobile WIMAX or Long Term Evolution (LTE) advanced, and/or any other current or future communication network, standard, and/or system.

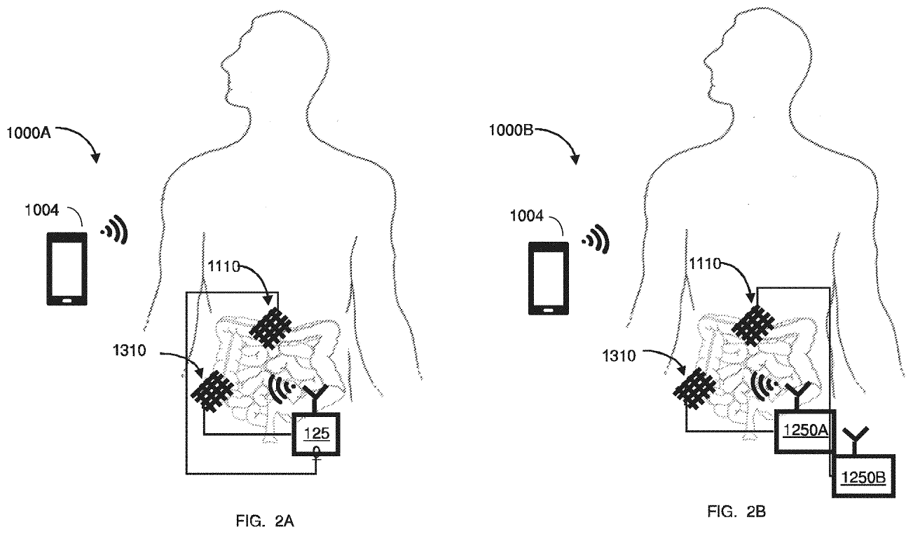

Reference is now made to FIGS. 2A and 2B, schematically illustrating various embodiments of a patient site of interest monitoring system 1000. One embodiment of patient site of interest monitoring system 1000 is herein designated by alphanumeric reference “1000A”, and another embodiment of monitoring system 1000 is herein designated by alphanumeric reference “1000B”. Merely to simplify the discussion that follows, without be construed in a limiting manner, the description may refer to “monitoring system 1000”.

As shown in FIGS. 2A and 2B, patient site of interest monitoring system 1000 includes a tissue-of-interest or SOI monitoring electrode 1110 that is operably coupleable adjacent to or such to engage with a site of interest internal to an animal (e.g., human) body, herein also referred to as “patient body”. Such site of interest may include, for example, an internal organ, a surgical intervention site (e.g., a tissue reconnection site such as an anastomosis site, a sleeve gastrectomy site, etc., a site which is prone to wound dehiscence or other tissue breakdown (e.g., due to tissue reconnection by employing, for example, staples, sutures, etc.), a hernia closure site, and/or any other physiological phenomenon expected or suspected to occur at the site of interest, and which physiological phenomenon may, for example, cause a measurable change in a characteristic (e.g., an electrical property) of a monitoring electrode, e.g., due to corrosion, (bio-)degradation, mechanical failure of the electrode(s) and/or the like. For example, a monitoring electrode may start bio-degrading or its biodegradation profile or its physical properties may otherwise change (e.g., accelerate or decelerate) as a result of and, e.g., in correspondence, with the onset or occurrence of a physiological phenomenon being monitored. Hence, occurrence and timing of the physiological phenomenon at the site of interest may be measurable by detecting a change in one or more characteristics of the monitoring electrode.

In some embodiments, tissue-of-interest monitoring electrode 1110 may, for example, be implantable in proximity of operably engaged with an anastomosis site of the GI track or any other surgical tissue connection site or other internal body site of interest, and may be implanted during Gastrointestinal (GI) surgery, for example. In some other embodiments, tissue-of-interest monitoring electrode 1110 may be operably integrated with a device extending from the internal body site of interest (or vicinity thereof) inside the patient's body to the outside of the patient's body, e.g., to the vicinity of the patient's skin surface. Such device may include, for example, a fluid drainage catheter that is operably positioned to drain fluids from the internal site of interest to the outside of the patient's body.

In some embodiments, a monitoring electrode 1110 and/or reference electrode 1310 may be biodegradable and/or biocompatible in full or in part. In some embodiments, a partially biodegradable monitoring electrode(s) 1110 and/or reference electrode(s) 1310 may comprise at least two parts, namely, a fully biodegradable part for engaging with tissue of a site of interest until the biodegradable part is degraded, and a partially bio-degradable or non-biodegradable part that can be retracted or removed after the monitoring period is completed. In some embodiments, a reference electrode is non-biodegradable.

In some embodiments, tissue-of-interest monitoring electrode 1110 may be biodegradable in full or in part yet not necessarily implantable (e.g., biocompatible) if it is designed to be operably positioned outside the patient's body, e.g., inside or as part of the fluid drainage catheter. For example, monitoring electrode 1110 may be operably coupled (e.g., embedded in the fluid drainage catheter such that the monitoring electrode can make contact with fluid flowing in the fluid path of the fluid drainage catheter.

A reference electrode 1310 may be employed for deployment at a different site of interest to provide an output that can be used as reference to the outputs provided by tissue-of-interest monitoring electrode 1110. Reference electrode 1310 may also be implantable, biocompatible and optionally biodegradable in full or in part. Reference electrode 1310 may for example be employed to improve accuracy of a specificity test, provide reference for spatial or temporal-spatial propagation of monitoring electrode dynamic electrical characteristics and/or of other methods that may be employed for performing diagnostic tests. In implementations where tissue-of-interest monitoring electrode 1110 is incorporated in a fluid drainage catheter, reference electrode 1310 may be located downstream and/or upstream of tissue-of-interest monitoring electrode 1110. The terms “upstream” and “downstream” as used herein in the context of a drainage catheter refers to a fluid drainage direction. In some embodiments, reference electrode 1310 may be deployed in a different, “reference” catheter that is operably coupled with the patient. Optionally, reference electrode 1310 may be biodegradable (fully or partially) or non-biodegradable and, in addition, not necessarily biocompatible), for example, if it is designed to be operably positioned outside the patient body, e.g., inside or as part of another fluid drainage catheter.

In some embodiments, signals received from the reference electrode may be used for performing self-calibration of components of the patient site of interesting monitoring system. For example, signals received from the reference electrode may be used for adapting at least one adverse-phenomenon output criterion based on which, for example, an output is provided for indicating that anastomotic leak occurs or not.

In some embodiments, monitor 1004 may provide artificial intelligence (including, e.g., machine learning) functionalities for adaptively changing the at least one adverse-phenomenon output criterion. The artificial intelligence functionalities may be based on patient-related characteristics.

As shown in FIGS. 2A and 2B, monitoring system 1000 may optionally include one or more communication devices 1250 for communicably linking between tissue-of-interest monitoring with a monitor 1004 and further for communicably linking reference electrodes 1310 with monitor 1004.

Communication device 1250 can include an active and/or a passive transmitter, receiver and/or transceiver. In some embodiments, communication device 1250 can be selectively switchable from an active to passive transmission mode and vice versa. Some or all components of communication device 1250 may be fully or partially biodegradable.

Monitor 1004 may incorporate functionalities of analysis engine 1260 and of output device 1240 which were outlined with respect to FIG. 1.

In patient site of interest monitoring system 1000A, a communication device 1250 may be communicably coupled with tissue-of-interest monitoring and reference electrodes 1110 and 1310. In another example of monitoring system 1000B, a first communication device 1250A may be communicably coupled with tissue-of-interest monitoring electrodes 1110, and a second communication device 1250B may be communicably coupled with reference electrodes 1310. First and second communication devices 1250A and 1250B may be communicably linked with each other in a wired and/or wireless communication to allow for in-body transmission of signals between the communication devices. In-body signal transmission may be employed to implement, for example, calibration, feedback, data fusion, noise-reduction, and/or other signal processing and/or analysis applications, for example, by analysis engine 1260 implemented by one or more implanted processors and/or memories.

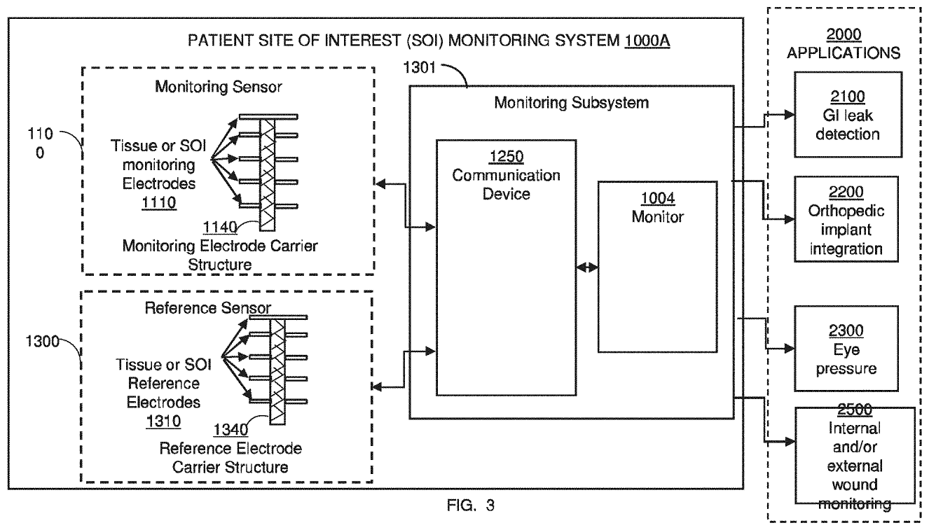

As schematically shown in FIG. 3 and mentioned herein with respect to FIGS. 2A and 2B, components of a monitoring subsystem 1301 may be configured to implement a communication device 1250 and a monitor 1004 to controllably subject tissue-of-interest monitoring with input signals via tissue-of-interest (TOI) monitoring and reference electrodes 1110 and 1310, respectively, with input or stimulation signals, and is further operable to communicate response signals relating to the input signals and which are received from tissue-of-interest monitoring and reference electrodes 1110 and 1310, respectively, to monitor 1004 which may be located outside the patient body.

For example, one or more tissue-of-interest monitoring electrodes 1110 and, optionally, one or more reference electrodes 1310, may be in communication with communication device 1250. Communication device 1250 may be communicably linked (wired and/or wirelessly) with monitor 1004 for providing monitor 1004 with signals generated by tissue-of-interest monitoring electrodes 1110 and, if applicable, by reference electrodes 1310. Monitor 1004 may be operable to process signals received from tissue-of-interest monitoring electrodes 1110 and, if employed or applicable, may also be operable to process signals received from reference electrodes 1310.

Monitor 1004 is operable to automatically or semi-automatically provide, based on the received response signals, an output pertaining to a physiological phenomenon (e.g., an or potentially adverse physiological phenomenon or condition) that may, for example, adversely affect the patient's health (e.g., wound dehiscence, anastomotic leakage, etc.) such as, for example, a warning upon detection of anastomotic leakage.

Response signals sent to monitor 1004 may be descriptive of, for example, impedance, capacitance, current flow and/or change thereof in response to detected changes of a physiological condition of the patient. In some embodiments the response signal sensed by any of the electrodes can be communicated based on the principle of electromagnetic induction. For example, patient site of interest monitoring system 1000 may comprise an implanted or implantable conductive coil (not shown). The implantable conductive coil may be part of communication device 1250, for example. Leak detection may further comprise an external conductive coil (not shown). The external coil may be part of or otherwise be operably coupled with monitor 1004. The internal and external coil are positioned or are operably positionable relative to each other such that a change in current in one coil is picked up by the other coil through electromagnetic induction. The internal coil may be biocompatible and can be partially or fully biodegradable. In some embodiments, the internal coil may be responsive to changes in the patient's physiological characteristics. For exam ple, the internal coil biodegrades or biodegrades at an accelerated or slower pace when being subjected to matter or (e.g., biological) substance that is a manifestation in a physiological phenomenon occurring in the patient's body. For example, characteristics (e.g., conductive or other electric characteristics) of the internal coil may change when coming in contact with fluid leaking from the GI tract. Changes in the internal coil's electric characteristics may manifest themselves in measurable variations of the electrical output signals that are output by the external coil. The implantable internal coil may thus embody a monitoring electrode, and the internal/external coil arrangement may embody an electronic sensor for detecting GI leak, for example.

Monitor 1004, which may be handheld, may comprise circuitry (e.g., a memory and a processor) for processing and/or analyzing response signals received from tissue-of-interest monitoring electrode 1110 and, optionally, of reference electrode 1310) to determine, based on the received signals, an output pertaining to a physiological condition of the site of interest internal to the patient body.

For example, monitor 1004 may be operable to employ a bandpass filter on the received signals. For instance, if electrodes are subjected to a stimulation input signal at 31 Hz, response signals may be bandpass or lowpass filtered at 0.5-15 Hz. The band pass filter on the received signals recorded from the site of interest can be adaptively calibrated to consider characteristics of the signals recorded from the reference site for a computationally efficient reference-based condition detection algorithm.

For example, monitor 1004 may determine, based on the received signals, if leakage occurs or not. For instance, monitor 1004 may provide, based on the received signals, a warning if characteristics of the received signals meet at least one adverse-phenomenon output criterion such as, for example, an adverse leak warning output criterion (e.g., an output indicating that anastomotic leak occurs); an output indicative of the likelihood or probability of an adverse physiological phenomenon leak to occur within a certain time period (i.e., predicting the onset of anastomotic leak); provide an output indicative of how a current treatment condition should be altered such to reduce (e.g., minimize) the likelihood or probability of an adverse physiological phenomenon (e.g., condition or state) to occur or develop, or prevent occurrence of anastomotic leak; provide an output indicative of supplementary diagnostic test to be performed for determining if leakage occurs or not; and/or the like.

Such adverse-phenomenon output criterion may pertain to sensed variations in electrical signal characteristics and may be static, a dynamic or adaptive leak-related output criterion. A static criterion is predetermined that remains constant. A dynamic criterion is forcefully changed, for example, at a certain time of day, or a certain day of the year. An adaptive criterion is changed in response to changes in, for example, physiological characteristics of the patient body and/or the patient body's environment, and may vary depending on a variety of parameters.

For example, a leakage warning may be provided if monitor 1004 detects a voltage drop below or voltage increase above a corresponding “low or high electrical characteristic warning threshold”; and/or if monitor 1004 detects a drop below or increase above a corresponding “low or high electrical characteristic warning threshold range”. The term “threshold” as used herein may refer to a predetermined threshold, a reference calibrated threshold, a moving threshold (e.g., linearly and/or non-linearly moving threshold) and/or any combination thereof. An adverse-phenomenon output criterion may in some implementations additionally or alternatively relate to a threshold relating to electrophysiological (e.g., electrogastrography) signal variance and/or other signal characteristics. For example, lower cross-correlation between electrophysiological signals recorded from the reference site vs. cross-correlation between signals recorded in the monitoring site may be indicative of increased likelihood of onset of anastomotic leak and/or provide indication of an anastomotic leak occurring. Optionally, analysis engine 1260 takes into account food intake when taking into account electrophysiological to determine if an adverse-phenomenon output criterion is met.

For example, a leakage warning may be provided if monitor 1004 detects an impedance drop below or impedance increase above a corresponding “low or high electrical characteristic warning threshold”; and/or if monitor 1004 detects a drop below or increase above a corresponding “low or high electrical characteristic warning threshold range”.

In certain embodiments, monitor 1004 and/or communication device 1250 may be operable to communicate with additional computing devices for providing expanded range of monitoring. Such computing device can include, for example, a multifunction mobile communication device also known as “smartphone”, a personal computer, a laptop computer, a tablet computer, a server (which may relate to one or more servers or storage systems and/or services associated with a business or corporate entity, including for example, a file hosting service, cloud storage service, online file storage provider, peer-to-peer file storage or hosting service and/or a cyberlocker), personal digital assistant, a workstation, a wearable device, a handheld computer, a notebook computer, a vehicular device and/or a stationary device.

In some embodiments, patient site of interest monitoring system 1000 is operative at low currents which are considered or known to be safe to the patient such as, for example, below 10 mA. For example, patient site of interest monitoring system 1000 is operative at low currents below low-threshold limits in the sense that such currents will have minimal effect on nerves or muscles including, for example, electrical currents below 1 mA (e.g., for muscles), or with currents below, for example, 10 nA (e.g., for nerves). In some embodiments, narrow current pulses may be utilized known to have reduced, minimal or no effect on muscles such as pulse widths shorter than, for example, 1 ms or pulse widths which are shorter than, for example, 100 us or other pulse widths known to have reduced, minimal or no effect on, e.g., human nerves, such as pulse widths shorter than, for example, 100 us. Low-threshold effects may be assured by applying currents at sufficiently low pulse widths. In some embodiments, a pulse width may exceed low-thresholds pulse-width limit, if the applied current is sufficiently low. In some embodiments, the applied current may exceed sub-threshold current limits if the pulse width with the currents are applied is sufficiently narrow. In some embodiments, electric pulses applied may be applied at frequencies which are below a corresponding low-threshold frequency limit.

Additional reference is made to FIG. 3. In some embodiments, one or more tissue-of-interest monitoring electrodes 1110 and reference electrodes 1310 may be operably coupled to a monitoring electrode carrier structures 1140 and a reference electrode carrier structure 1340, respectively. The electrode carrier structures facilitate securely and operably engaging (e.g., operably non-removably or removably coupling or fastening) the tissue-of-interest monitoring and reference electrodes 1110 and 1310, respectively, with organ tissue.

Optionally, the one or more TOI monitoring electrodes 1110 may herein be referred to in the singular as a “tissue-of-interest monitoring sensor 1100”, and the one or more TOI reference electrodes 1310 may be referred to in the singular as a “reference sensor 1300”. Optionally, the one or more tissue-of-interest monitoring electrodes 1110 and a tissue-of-interest monitoring electrode carrier structure 1140 to which the one or more tissue-of-interest monitoring electrodes 1110 are coupled, may herein be referred to as a “tissue-of-interest monitoring sensor 1110”. Analogously, the one or more reference electrodes 1310 and a reference electrode carrier structure 1340 to which the one or more reference electrodes 1310 may be coupled may herein be referred to as “reference sensor 1300”. Such (leak or reference) electrode carrier structure can be in the form of a mesh, for example. In some embodiments, tissue-of-interest monitoring electrodes 1110 may be operably coupled with monitoring electrode carrier structure 1140 to form a tissue-of-interest monitoring sensor 1100. Analogously, reference electrodes 1310 may be operably coupled with a reference electrode carrier structure 1340 to form reference sensor 1300.

In some embodiments, material(s) that are used for constructing a mesh may constitute a part of tissue-of-interest monitoring and/or reference electrodes 1110 and 1310. In some embodiments, tissue-of-interest monitoring and/or reference electrodes 1110 and 1310 may be arranged to form, respectively, tissue-of-interest monitoring and reference carrier structures 1140 and 1340.

In some embodiments, sensing elements of an (e.g., implantable) electronic sensor such as the monitoring electrode and/or a mesh may have a material thickness and/or other properties to ensure detectability of changes of a physiological phenomenon in a patient. The sensing element(s) may be implantable or non-implantable. Optionally, a part of a sensing element may be implantable, and a part may be non-implantable. Optionally, a part of the sensing element may be biodegradable, and another part may be non-biodegradable. For example, to attain a detectable corrosion rate for a desired period of time while retaining mechanical properties of the monitoring electrode, for instance, a metal alloy wire of the monitoring electrode may have a diameter ranging, for example, from, 100 μm to 800 μm. In this case, analysis engine 1260 may consider the non-linear corrosion pattern of the electrode and its effect on the sensor's impedance. It is noted that the electrode or components thereof may include a cable, a sheath metal, a film, a single wire, and/or the like. The sensing elements may include one or more electrodes, meshes, rods, strings, wires, cables, machined sheath metals, film, and/or the like.

The term “mesh” as used herein, may refer to a two- or multidimensional semipermeable structure of closely-spaced holes, which is composed of a plurality of elongated and interconnected elements, such as fibers, strands, struts, spokes, rungs made of a flexible/ductile material, which are arranged in an ordered (matrix, circular, spiral) or random fashion to form e.g., a two-dimensional sheet or a three-dimensional object. In some embodiments, the term “mesh” is intended to include an element having an openwork fabric or structure, and may include but is not limited to, an interconnected network of wire-like segments, a sheet of material having numerous apertures and/or portions of material removed, or the like. Accordingly, the term “mesh” may also refer to a matrix or a net structure. A wire-like segment may for example comprise monofilaments and/or braided fibers. In some embodiments, the mesh may comprise or embed one or more electrodes.

In some embodiments, by “closely-spaced holes” it is meant to refer to a spacing of e.g., 1 mm, 2 mm, 5 mm, 10 mm, 15, mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, or 200 mm, including any value and range therebetween.

According to some embodiments, certain meshes may be composed of fibrous elements which come in direct physical contact with each other at each intercrossing junction constituting the mesh.

In some embodiments, tissue-of-interest monitoring electrodes 1110, reference electrodes 1310, monitoring electrode carrier structure 1140 and/or electrode carrier structure 1340 comprise or are made of conductive, biocompatible and/or (bio-) degradable material(s). For example, the mesh or the wire structure comprises a core structure coated with conductive, biocompatible and/or biodegradable material(s). In some embodiments, the core comprises one or more metals. Optionally, the mesh structure comprises non-conductive polymer fibers that are interwoven with conductive, biocompatible and/or biodegradable material(s). Optionally, the wire (or the mesh) has a uniformly porous architecture so that degradation of the wire (or the mesh) can be progressed uniformly. Optionally, the mesh has a dimension of 0.1 mm to 500 mm×0.1 mm to 500 mm, including any value and range therebetween.

Tissue-of-interest monitoring electrodes 1110, reference electrodes 1310, monitoring electrode carrier structure 1140 and reference electrode carrier structure 1340 can be made of any suitable material including, for example, non-biodegradable, partially biodegradable, or fully biodegradable material such as, for example, magnesium-based material, magnesium alloys, stainless steel, carbon tip, platinum, platinum-iridium alloy, gold, and/or any type of biodegradable and/or biocompatible alloy or metal. Tissue-of-interest monitoring electrode carrier structures 1140 and reference electrode carrier structures 1340 may for example comprise, in addition to the metal components, Vicryl, Polyglycolicacid:Trimethylene (PGA:TMC), PGA, and/or non-(bio)degradable materials that are employable in implantable devices, and/or any other suitable material.

Mg impedance for example is increasing due to the generation of corrosion products (metal oxides and metal hydroxides) covering some of the effective surface area of the electrode. Due to corrosion, effective electrode surface area is changing, mainly decreasing, thus making the conductive contact area of the electrode with the tissue smaller.

Tissue-of-interest monitoring electrode carrier structure 1140 and reference electrode carrier structure 1340 may be operably fastened to organ tissue by employing, for example, various fixation elements and/or methods including, e.g., glue, adhesives and/or sutures. For example, barbed Polyglyconate biodegradable suture may be threaded into a mesh to facilitate local fastening and for stabilizing monitoring sensor 1100 and reference sensor 1300 in place of corresponding sites of interest, without puncturing or otherwise inadvertently damaging tissue structure such as the GI tract.

As noted, monitor 1004 comprises circuitry (e.g., a processor and a memory) that is configured to adaptively change at least one adverse-phenomenon output criterion. The at least one adverse-phenomenon output criterion may be changed adaptively based on, for example, patient-related characteristics deemed relevant to proper evaluation of patient recovery. Patient-related characteristics can include, for example, information about the patient's medical history (e.g., type(s) of surgical GI procedure(s) which the patient underwent); social characteristics (e.g., gender; age; profession; income), and/or the like. Patient-related characteristics may also include measured physiological characteristics such as, for example, biometric data; systolic blood pressure; diastolic blood pressure; mean arterial pressure; pulse rate; breathing rate; breathing pattern; oxygen saturation level; glucose level; electrical property of the patient's skin (e.g., conductivity, resistance); weight, body-mass index (BMI) pH level; concentration of one or more selected analytes in bodily fluid (e.g., magnesium, calcium, sodium, salts, glucose, and/or hormones); motor function; body temperature; sweat rate; electrocardiogram; myocardiogram; electroencephalography; capnography values; a cognitive ability of the patient; and/or the like. Bodily fluid can include blood, sweat, tears and/or saliva. In some embodiments, environmental parameters may be taken into account which can include, for example, location, temperature, humidity, room particle count, pressure level of the environment in which the patient is located and/or the like. In some embodiments, patient-related characteristics may pertain to third party data descriptive of information that may be deemed relevant for assessing patient recovery. Such third party data include data of other patients, data associated with relatives of the patient, and/or the like.

Although, embodiments of the present application pertain to the detection of anastomotic leak, the systems and methods disclosed herein may also be applied in the monitoring of various other health applications in which internal (e.g., adverse) physiological phenomena effecting electronic parameters or characteristics like impedance, phase shift, capacitance, and/or current flow voltage potential, can be rendered into electronic data. In particular the system and method described herein translates physiological conditions into data, allowing close monitoring of a surgical site for healing and recovery, and early detection of complications, namely inflammatory and/or infectious complications. Various example set forth in under “applications” 2000, include, inter alis, lumen leak detection 2100, orthopedic implant integration 2200, eye pressure 2300, and/or (e.g., head, sternum) other internal and/or external wound monitoring 2500. Further applications 2000 may include, for example, monitoring healing; post tumor resection monitoring of organs with respect to recurrence; monitoring indications related to signs of rejection of transplanted organs to adjust medication such as immune-depressive pharmacological therapy dosing; monitoring for infections adjacent to any foreign surgical implants; monitoring high-risk surgical wounds (e.g., contaminated wound areas, groin, trauma wounds; monitoring areas of lymphatic dissections; monitoring areas of lymph node resections; monitoring urinary bladder function; monitoring stomach, small bowel and/or large bowel functions; monitoring sphincters including, for example, lower esophageal sphincter, pylorus, anal sphincter, and/or the like; monitoring vascular functions including, for example, renal artery and/or carotid tone monitoring; monitoring of the peritoneum, e.g., for detecting postoperative peritonitis; monitoring the mediastinum and/or the pleural cavity for postoperative mediastinitis and pleuritis and/or empyema; and/or the like.

It should be noted that while certain aspects of the present disclosure pertains to the detection of GI leaks, this should by no means be construed in a limiting manner. Accordingly, devices, systems and methods described herein may also be employed for detecting other physiological phenomenon occurring at or in the vicinity of a site of interest and which can have a measurable effect on electrical characteristics of monitoring electrodes. For example, system and methods disclosed herein can be employed in the monitoring of internal organs such as the pancreas, liver or bladder to detect a leak, evaluate healing and/or changes in inflammatory and/or malignant conditions. Furthermore, a monitoring electrode can be implanted in a site in which eminent physiological changes are expected or suspected in order to detect occurrence timing of said changes or to provide an indication that such changes are not occurring as expected.

In some embodiments the rate of biodegradation of a part of entirety of the system will be set in line with the expected timing of expected leak or other said expected or suspected physiological changes. Specifically, in some embodiments the implantable parts in the vicinity of the internal body site of interest in which the leak or other physiological phenomenon is expected or suspected are designed to bio-degrade at a similar timing of the expected physiological phenomenon, whereas the other implantable parts that reach away from the said site may be made to be biocompatible but not biodegradable.

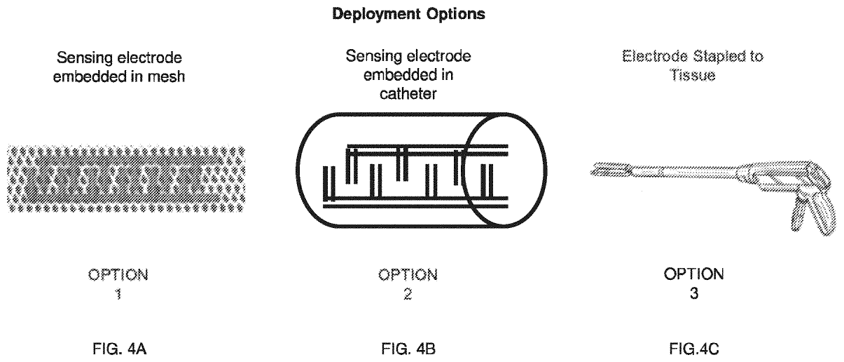

As shown in FIG. 4A, a first Option 1 is directed at placing a monitoring electrode in a mesh and operably fastening them to a site of interest for the monitoring thereof.

As shown in FIG. 4B, a second Option 2 is directed to embedding a sensing or monitoring electrode in a catheter, for example, a post-surgical drain catheter, to bring the sensing in contact with liquids that may be drained from the site of interest. Liquids drained from the site of interest may reflect a physiological condition such as, for example, tissue breakdown, wound dehiscence, which may manifest itself in anastomotic leak.

As shown in FIG. 4C, a third Option 3 is directed to securing the monitoring electrodes to an body site of interest with staples, e.g., as is known in the art.

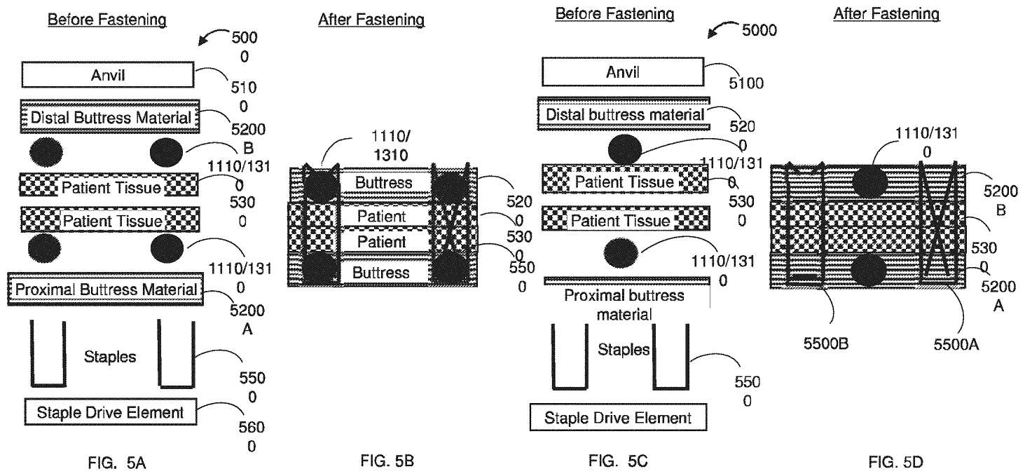

FIGS. 5A-5B depict different stages of stapling tissue-of-interest monitoring and/or reference electrode 1110/1310 of patient site of interest monitoring system 1000 into a deployment position in patient tissue 5300 with a surgical stapler 5000, according to some embodiments. Staple drive element 5600 is operable to drive staples 5500 (e.g., staples 5500A and 55008) against an anvil 5100 of surgical stapler 5000 subjecting, in turn, the application of a force onto staple 5500 causing the staple to contort upon contact with anvil 5100 such that the legs of the staple are bent within staple clinching pockets (not shown) of anvil 5100 relative to the staple's backspan while and/or after the legs traverse the patient's tissue to thereby secure distal buttress material 5200A against tissue 5300 and to secure the backspan to proximal buttress material 5200B against the patient's proximal tissue portion 5300.

As shown schematically in FIGS. 5A-5B, each staple 5500 may in some embodiments be applied in alignment with the monitoring electrode(s) such that the staple directly engages with the monitoring electrode(s) for the fastening thereof with the patient's tissue. Sensing/monitoring electrode may be present at both sides of the staple line, or only one side.

As shown schematically in FIGS. 5C-5D, the monitoring electrode(s) may in some embodiments be positioned between two staples 5500 such that the monitoring electrodes are secured to the patient's tissue merely by the force the staples apply for securing the proximal and distal buttress materials to the tissue. For example, the monitoring electrodes are not directly fastened onto the patient tissue but secured through the fastening of the buttress material. In some embodiments, no buttress material may be employed. It should be appreciated that in certain deployments a circular stapler may be employed. A swine model was employed in which retractrable and resorbable monitoring electrodes were implanted and engaged with a leak site at a descending colon location, and in which also retractable and resorbable reference electrodes were implanted at a reference site in the ascending/spiral colon.

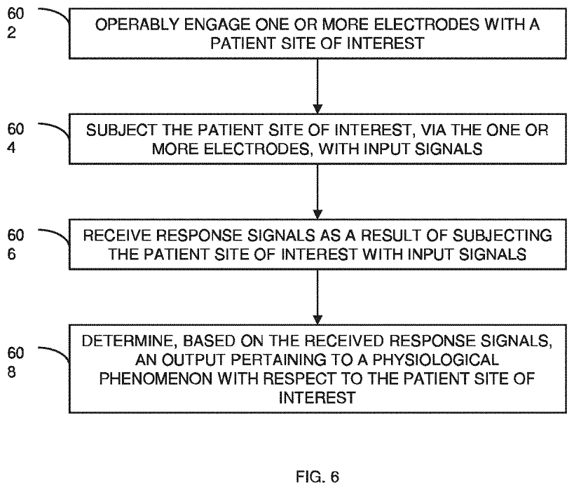

Further referring to FIG. 6, a method of monitoring a patient site of interest comprises, according to some embodiments, operably engaging one or more electrodes with a patient site of interest (block 602).

In embodiments, the method may further comprise subjecting the patient site of interest, via the one or more electrodes, with input signals (block 604).

In embodiments, may comprise receiving response signals as a result of subjecting the patient site of interest with input signals (block 606).

In embodiments, the method may further comprise determining, based on the received response signals, an output pertaining to a physiological phenomenon with respect to the patient site of interest (block 608).

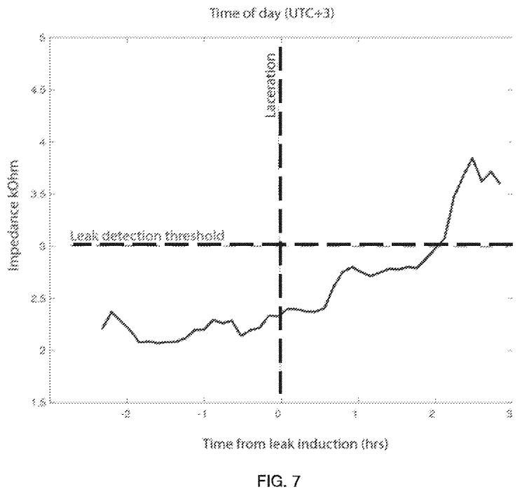

Referring now to FIG. 7, the solid line shows impedance as a function of time. The horizontal dashed line indicates a leak detection threshold and the vertical dashed line indicates a time of laceration. When measured impedance crosses the threshold for a certain period of time (e.g., >10 min), an output indicative of anastomotic leak is provided. The threshold can for example be fixed, predetermined, or can change in a linear or a non-linear manner.

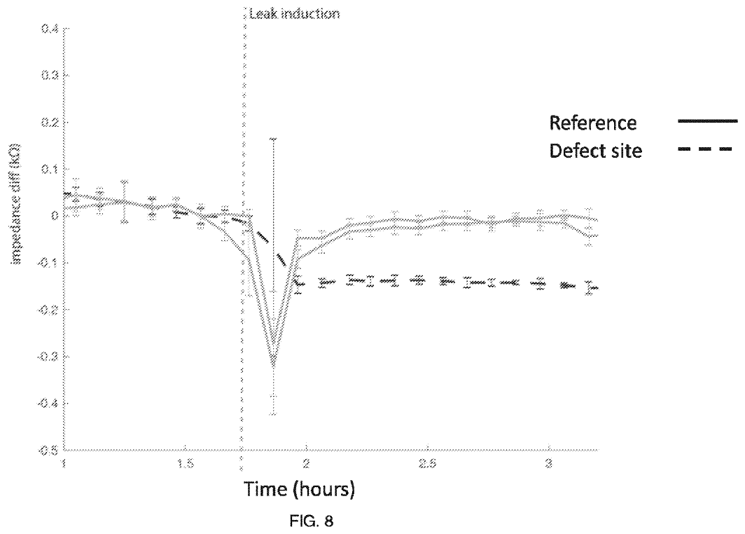

Further reference is made to FIG. 8 in which the dashed line indicates the impedance difference from baseline measured 10 mm from the leak site prior and after the induction of the defect. The sold line indicates impedance of two reference electrodes recorded at a distance >100 mm from the defect. The error bars represent standard deviation of impedance measured over 2 minutes per sample. Overall separation 10 minutes after the induction of the defect reflects p<0.001

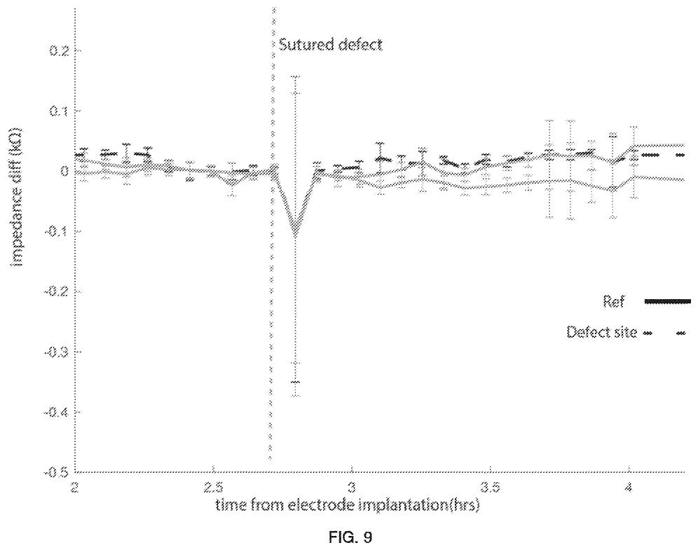

FIG. 9 shows impedance measurement in an animal with a sutured defect (no leak). In this case there is no separation over time between the recorded impedance before and after the induction of the leak.

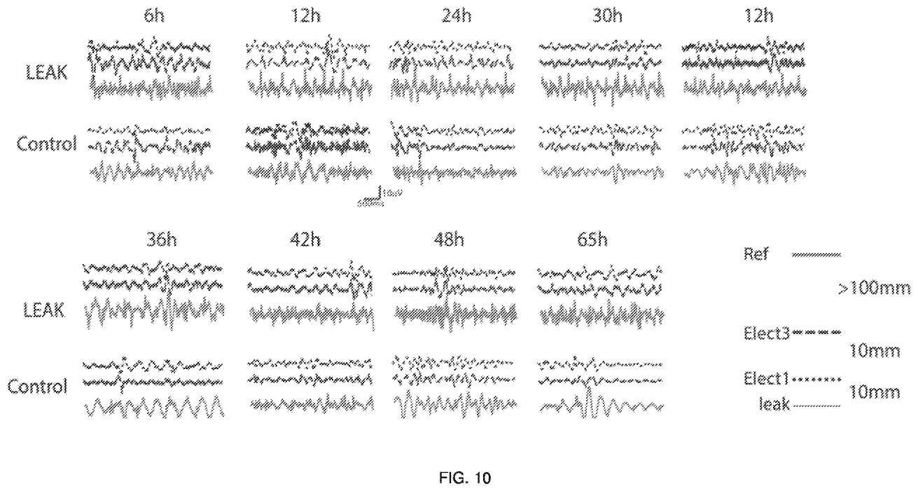

FIG. 10 show representative electrophysiological signals from electrodes implanted 10 mm, 20 mm and >100 mm from the defect in a chronic leak animal model and control animal model with healing defect. It is noted that there is typically better correlation between the reference and defect signals recorded in the control animal compared to the leak animal model.

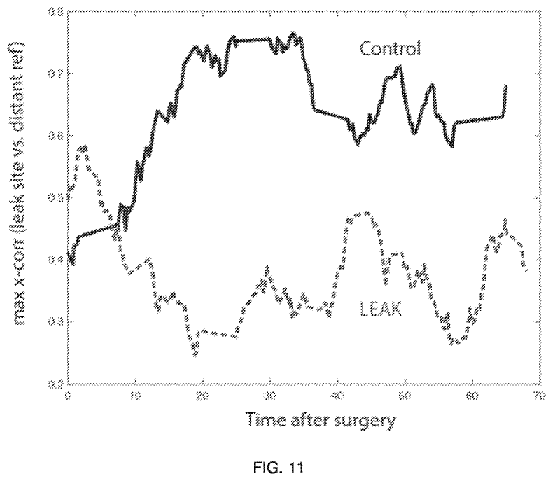

FIG. 11 shows maximum cross correlation value (“correlation coeff”) of leak animal model vs. control animal model over time. The leak model shows declining correlation between the healthy reference site and the damaged site while the correlation improves on the control model, in line with the physiological condition in the respective animals.

### ADDITIONAL EXAMPLES

Examples pertain to a monitoring system for detecting or predicting the onset of adverse physiological phenomenon in a patient, the system comprising an electronic sensor having electrical characteristics that are responsive to changes in physiological characteristics of a patient with which the electronic sensor is operably coupled, the electronic sensor being operable to provide an output descriptive of changes in physiological characteristics of the patient; and processing circuitry that is configured to process the output to determine if the output meets at least one adverse-phenomenon output criterion or not. The output can include an electronic or otherwise measurable signal.

Example 1 includes a monitoring system for detecting or predicting the onset of adverse physiological phenomenon in a patient, the monitoring system comprising an electronic sensor having electrical characteristics that are responsive to changes in physiological characteristics of a patient with which the electronic sensor is operably coupled; a communication device that is in communication with the electronic sensor and operative to receive an electric signal from the electronic sensor, the electric signal being indicative of changes in the physiological characteristics of the patient, wherein the communication device is further operative to transmit (wired and/or wirelessly) a signal relating to the received electric signal; and processing circuitry that is configured to process the received electric signal to determine if the received electric signal meets at least one adverse-phenomenon output criterion or not.

Example 2 includes the subject matter of Example 1 and, optionally, wherein the processing circuitry is configured to process the received signal to determine if anastomotic leakage occurs or not.

Example 3 includes the subject matter of examples 1 or 2 and, optionally, wherein the electronic sensor comprises an implantable electrode.

Example 4 includes the subject matter of Example 3 and, optionally, wherein the electronic sensor comprises a sensing element that includes, for example, an implantable mesh, wire, sheath metal and/or cable.

Example 5 includes the subject matter of Examples 4 and, optionally, wherein the sensing element comprises biodegradable material.

Example 6 includes the subject matter of any one or more of the Examples 4 or 5 and, optionally, wherein the sensing element comprise biocompatible material.

Example 7 includes the subject matter of any one or more of Examples 3 to 6 and, optionally, wherein the sensing element comprises biocompatible material and which is deployable and coupleable adjunct to the site of interest.

Example 8 includes the subject matter of any one or more of Examples 1 to 7 and, optionally, further comprising a fluid drainage catheter that is operably positionable to drain fluids from an internal body site of interest to outside the body of the patient, wherein the electronic sensor is operably integrated in and/or fluidly coupled with the fluid path of the bodily fluid drainage catheter for measuring changes in the physiological characteristics of the patient.

Example 9 comprises a patient site of interest monitoring system for detecting or predicting the onset of adverse physiological phenomenon in a patient, the system comprising: a monitoring sensor for operably engaging a patient site of interest, the monitoring sensor having electrical characteristics that are responsive to changes in physiological characteristics of the patient site of interest; an input device for generating and subjecting the patient of interest with an input signal via the monitoring electrode to generate a response signal; a communication device that is in communication with the monitoring sensor and operative to receive the response signal from the electronic sensor, the response signal being indicative of changes in the physiological characteristics of the patient; and an analysis engine that is configured to receive the response signal from the communication device and to process data relating to the received response signal to determine if the response signal meets at least one adverse-phenomenon output criterion or not.

Example 10 includes the subject matter of example 9 and, optionally, wherein the analysis engine is configured to process the received signal to determine if leakage from a body organ occurs or not.

Example 11 includes the subject matter of examples 9 and/or 10 and, optionally, wherein the monitoring sensor comprises a sensing element.

Example 12 includes the subject matter of any one or more of the Examples 9 to 11, wherein the monitoring sensor is fully implantable, partially implantable, or non-implantable monitoring sensor, and, optionally, a reference sensor that is a fully implantable, partially implantable, or non-implantable reference sensor.

Example 13 includes the subject matter of Examples 11 and/or 12, wherein the sensing element comprises biodegradable material.

Example 14 includes the subject matter of Examples 12 and/or 13 and, optionally, wherein the sensing element comprises biocompatible material.

Example 15 includes the subject matter of any one or more of the examples 12 to 14 and, optionally, wherein the monitoring sensor comprises biocompatible material.

Example 16 includes the subject matter of any one or more of the examples 9 to 15, and, optionally, further comprising a fluid drainage catheter that is operably positionable to drain fluids from an internal body site of interest to outside the body of the patient, wherein the monitoring sensor is operably integrated in the fluid path of the bodily fluid drainage catheter for measuring changes in the physiological characteristics of the patient.

Example 17 includes the subject matter of Example 16 and, optionally, wherein the monitoring sensor is operably integrated in the fluid path of the bodily fluid drainage catheter for measuring changes in the physiological characteristics of the patient site of interest.

Example 18 includes the subject matter of any one or more of the Examples 9 to 17 and, optionally, wherein the input device is operable to subject the patient site of interest with an alternating input signal at an automatically or manually selected input frequency

Example 19 includes the subject matter of Example 18 and, optionally, wherein the alternating input signal is pre-selected, dynamically selected or adaptively selected.

Example 20 includes the subject matter of Examples 18 and/or 19 and, optionally, wherein the input device is operable to subject the patient site of interest with an alternating input signal at a plurality of automatically or manually selected frequencies.

Example 21 includes the subject matter of any one or more of the Examples and, optionally, 9 to 20, further comprising a reference sensor for providing a reference signal, the reference sensor optionally comprising a fully implantable, partially implantable, and/or non-implantable sensing element.

Example 22 includes the subject matter of any one or more of the examples 9 to 21 and, optionally, wherein a characteristic of the input signal is selected automatically based on the reference signal.

Example 23 includes the subject matter of Example 22 and, optionally, wherein a characteristic of the input signal and/or response signal comprises amplitude, frequency and/or phase of the signal.

Example 24 includes the subject matter of Example 23 and, optionally, wherein a characteristic of the input signal further comprises input signal and/or response signal filter characteristics.

Example 25 includes the subject matter of Example 24 and, optionally, wherein the input and/or response signal filter characteristics are predetermined, dynamically selected or adaptively selected.

Example 26 includes the subject matter of any one or more of examples 20 to 25, and, optionally, wherein the input device is operable to sweep a plurality of frequencies of the input signal.

Example 27 includes the subject matter of Example 26 and, optionally, wherein the sweeping is performed through a predetermined range, a dynamically selected or adaptively selected frequency range.

Example 28 includes the subject matter of any one or more of the Examples 9 to 27 and, optionally, wherein when an output that is provided by the monitoring sensor has characteristics meeting at least one adverse-phenomenon output criterion, then the output is indicative of anastomotic leak. The at least one adverse-phenomenon output criterion may, for example, relate to a measured variation in impedance such as, for example, increase above a threshold. Optionally, the monitoring sensor output may be compared against a reference sensor output, and the at least one adverse-phenomenon output criterion may pertain to a result of the comparison between the two outputs. For example, a difference between a monitoring sensor outputs value and a reference sensor output value exceeding a threshold may be indicative of anastomotic leak.

Example 29 includes the subject matter of any one or more of the Examples 9 to 28 and, optionally, further comprising a reference sensor.

Example 30 includes the subject matter of any one or more of the Examples 9 to 20 and, optionally, wherein the monitoring and/or reference sensors include a monitoring and/or reference electrode, respectively.

Example 31 includes the subject matter of Example 30 and, optionally, wherein the monitoring and/or reference electrodes comprise Magnesium.

Example 32 includes the subject matter of any one or more of the Examples 9 to 31, and, optionally, wherein the monitoring and/or reference sensor is configured such that a change in measured impedance (e.g., change in pattern of measured impedance, is indicative of leak).

Example 33 includes the subject matter of any one or more of the Examples 9 to 32 and, optionally, wherein the analysis engine determines based on the electrophysiological signals if the response signal meets one or more adverse-phenomenon output criteria or not.

Example 34 includes the subject matter of any one or more of the Examples 9 to 33 and, optionally, wherein the analysis engine employs artificial intelligence functionalities for determining if the response signal meets one or more adverse-phenomenon output criteria or not.

Example 35 includes the subject matter of any one or more of the Examples 30 to 34 and, optionally, wherein the monitoring and/or reference electrodes are arranged in alignment with a staple such that the staple directly engages with the electrode for the fastening thereof with the patient's tissue.

Example 36 includes the subject matter of any one or more of the Examples 30 to 35 and, optionally, wherein monitoring and/or reference electrodes are positioned between two staples such that the electrodes are secured to the patient's tissue merely by the force of the staples for securing a proximal and distal buttress materials to the tissue.

Example 36 is method for monitoring a patient site of interest, comprising operably engaging one or more electrodes with a patient site of interest; subjecting the patient site of interest, via the one or more electrodes, with input signals; receive response signals as a results of subjecting the patient site of interest with input signals; and determining, based on the received response signals, an output pertaining to a physiological phenomenon with respect to the patient site of interest, wherein the physiological phenomenon pertains to anastomotic leak.

The various features and steps discussed above, as well as other known equivalents for each such feature or step, can be mixed and matched by one of ordinary skill in this art to perform methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

Any digital computer system, module and/or engine exemplified herein can be configured or otherwise programmed to implement a method disclosed herein, and to the extent that the system, module and/or engine is configured to implement such a method, it is within the scope and spirit of the disclosure. Once the system, module and/or engine are programmed to perform particular functions pursuant to computer readable and executable instructions from program software that implements a method disclosed herein, it in effect becomes a special purpose computer particular to embodiments of the method disclosed herein. The methods and/or processes disclosed herein may be implemented as a computer program product that may be tangibly embodied in an information carrier including, for example, in a non-transitory tangible computer-readable and/or non-transitory tangible machine-readable storage device. The computer program product may directly loadable into an internal memory of a digital computer, comprising software code portions for performing the methods and/or processes as disclosed herein. The term “non-transitory” is used to exclude transitory, propagating signals, but to otherwise include any volatile or non-volatile computer memory technology suitable to the application.

Additionally or alternatively, the methods and/or processes disclosed herein may be implemented as a computer program that may be intangibly embodied by a computer readable signal medium. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a non-transitory computer or machine-readable storage device and that can communicate, propagate, or transport a program for use by or in connection with apparatuses, systems, platforms, methods, operations and/or processes discussed herein.

The terms “non-transitory computer-readable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by one or more communication networks.

These computer readable and executable instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable and executable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable and executable instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

Unless otherwise specified, the terms ‘about’ and/or ‘close’ with respect to a magnitude or a numerical value may imply to be within an inclusive range of −10% to +10% of the respective magnitude or value.

“Coupled with” means indirectly or directly “coupled with”.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not to be construed as excluding an embodiment referring to a condition “equal or below a threshold”. It is clear that should a condition be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

It should be understood that where the claims or specification refer to “a” or “an” element and/or feature, such reference is not to be construed as there being only one of that element. Hence, reference to “an element” or “at least one element” for instance may also encompass “one or more elements”.

As used herein the term “configuring” and/or ‘adapting’ for an objective, or a variation thereof, implies using materials and/or components in a manner designed for and/or implemented and/or operable or operative to achieve the objective.

Unless otherwise stated or applicable, the use of the expression “and/or” between the last two members of a list of options for selection indicates that a selection of one or more of the listed options is appropriate and may be made, and may be used interchangeably with the expressions “at least one of the following”, “any one of the following” or “one or more of the following”, followed by a listing of the various options.

As used herein, the phrase “A,B,C, or any combination of the aforesaid” should be interpreted as meaning all of the following: (i) A or B or C or any combination of A, B, and C, (ii) at least one of A, B, and C; and (iii) A, and/or B and/or C. This concept is illustrated for three elements (i.e., A,B,C), but extends to fewer and greater numbers of elements (e.g., A, B, C, D, etc.).

As used herein, “biodegradable” materials include materials that at least partially resorb into the body or otherwise break down over time while not necessarily being absorbed within the body, and “non-biodegradable” materials include those that maintain substantial mechanical integrity over their lifetime in a body. Such “biodegradable” or “nonbiodegradable” materials are well known to those having skill in the art. In some embodiments, these materials will be biocompatible, while in other embodiments, they may be partially or fully constructed from non-biocompatible materials.

It is noted that the terms “operable to” or “operative to” can encompass the meaning of the term “adapted or configured to”. In other words, a machine “operable to” or “operative to” perform a task can in some embodiments, embrace a mere capability (e.g., “adapted”) to perform the function and, in some other embodiments, a machine that is actually made (e.g., “configured”) to perform the function.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It should be appreciated that combination of features disclosed in different embodiments are also included within the scope of the present inventions.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A monitoring system for detecting or predicting onset of adverse physiological phenomenon in a patient, the system comprising:
an electronic sensor operably coupleable with a patient site of interest and having electrical characteristics that are responsive to changes in physiological characteristics of the patient of site interest,
the electronic sensor being operable to provide an output signal descriptive of changes in physiological characteristics of the patient; and
processing circuitry that is operative to detect, based on the output signal, anastomotic leakage in the patient or onset of anastomotic leakage.

2-7. (canceled)

8. The monitoring system of claim 1, further comprising a fluid drainage catheter that is operably positionable to drain fluids from an internal body site of interest to outside the body of the patient, wherein the electronic sensor is operably integrated in or in fluid communication with a fluid path of the bodily fluid drainage catheter for measuring changes in the physiological characteristics of the patient.

9. A patient site of interest monitoring system for detecting or predicting onset of adverse physiological phenomenon in a patient, the system comprising:
a monitoring sensor for operably coupling with a patient site of interest, the monitoring sensor having electrical characteristics that are responsive to changes in physiological characteristics of the patient site of interest;
an input device for generating and subjecting the patient site of interest with an input signal via the monitoring sensor to generate a response signal;
a communication device operative to receive the response signal from the monitoring sensor, the response signal being indicative of changes in the physiological characteristics of the patient; and
an analysis engine that is operative to receive the response signal from the communication device and to process data relating to the received response signal to detect leakage from a body organ.

10-17. (canceled)

18. The patient site of interest monitoring system of claim 1, further configured to detect one of the following: tissue inflammations, tissue infections, tissue breakdown, tissue dihiscence or any combination of the aforesaid.

19. (canceled)

20. The patient site of interest monitoring system of claim 18, further configured to monitor a healing process.

21. The patient site of interest monitoring system of claim 9, further comprising a reference sensor for providing a reference signal;
wherein a characteristic of the input signal is selected automatically based on the reference signal.

22-25. (canceled)

26. The patient site of interest monitoring system of claim 20, wherein the input device is operable to sweep a plurality of frequencies of the input signal.

27. (canceled)

28. The patient site of interest monitoring system of claim 9, wherein when an output that is provided by the monitoring sensor has characteristics meeting one or more adverse-phenomenon output criteria, then the output relates to anastomotic leak or an onset thereof.

29. The patient of interest monitoring system of claim 9, further comprising a reference sensor.

30-31. (canceled)

32. The patient site of interest monitoring system of claim 9, wherein the monitoring and/or reference sensor is operative such that a change in measured impedance pattern is indicative of leak.

33. The patient site of interest monitoring system of claim 9, wherein the analysis engine determines based on the electrophysiological signals if the response signal meets one or more adverse-phenomenon output criteria or not.

34. The patient site of interest monitoring system of claim 9, wherein the analysis engine is configured to assess patient recovery following anastomotic leakage.

35-36. (canceled)

37. A method for monitoring a patient site of interest, comprising:
operably engaging one or more electrodes with a patient site of interest; subjecting the patient site of interest, via the one or more electrodes, with input signals; receiving response signals as a result of subjecting the patient site of interest with input signals; and
determining, based on the received response signals, an output pertaining to a physiological phenomenon with respect to the patient site of interest, wherein the physiological phenomenon pertains to anastomotic leak.

38. The monitoring system of claim 1, wherein the output signal relates to electrogastric activity.

39. The monitoring system of claim 1, wherein the output signals relates to food intake by the patient.

40. The monitoring system of claim 1, wherein the processing circuitry is operative to detect anastomotic leakage based on cross-correlation of physiological signals sensed at the patient site of interest and further based on cross-correlation of physiological signals sensed at a reference site different from the patient site of interest.

41. The monitoring system of claim 1, wherein the processing circuitry is operative to detect anastomotic leakage based on cross-correlation of physiological signals sensed at the patient site of interest and further based on cross-correlation of physiological signals sensed at a reference site different from the patient site of interest.

42. The monitoring system of claim 1, wherein the monitoring system comprises a plurality of electronic sensor configured to provide multiple signal outputs,
wherein the circuitry is configured to perform, based on the multiple signal outputs, temporal-spatial monitoring of the patient site of interest.

43. The monitoring system of claim 1, wherein the output signal is indicative of a change of an electrical property of the electronic sensor,
wherein the circuitry is configured to detect anastomotic leakage based on the change of the electrical property of the electronic sensor.

44. The monitoring system of claim 1, further comprising:
a first communication device that is operably coupled with the electronic sensor employed for monitoring the patient site of interest, wherein the electronic sensor and the other electronic sensor are implantable sensors; and
a second communication device that is operably coupled with another electronic sensor employed for monitoring a reference site which is at a different location than the patient site of interest, wherein the first and the second communication devices are configured to perform in-body transmission of signals between the electronic sensor and the other electronic sensor.
